(19) United States
(12) Patent Application Publication
(10) Pub. No.: US 2006/0122469 A1
(43) Pub. Date: Jun. 8, 2006

(54) Remote medical monitoring system

(76) Inventor: Normand M. Martel, E. Kingston, NH (US)

Correspondence Address: LAMBERT & ASSOCIATES, P.L.L.C., 92 STATE STREET, BOSTON, MA, 02109-2004, US

(21) Appl. No.: 10/990,690

(22) Filed: Nov. 16, 2004

(51) Int. Cl. A61B 5/00; A61B 19/00

(52) U.S. Cl. 600/300; 128/898

(57) ABSTRACT

A medical monitoring system that brings the hospital-campus telemetry experience to the patient home. This system is designed to enable effective step-down patient care in the home setting while providing the patient with the freedom to go anywhere and remain “logically” tethered to the system. The system achieves this by being distributed in nature, globally accessible, highly scalable, with near real-time concurrent reporting and analysis of multiple physiological parameters, and making this information real-time accessible to healthcare practitioners.

### BACKGROUND OF THE INVENTION

1. Field of the Invention

This invention relates generally to the field of medical monitoring systems and more particularly to a remote medical monitoring system, that is distributed in nature, is globally accessible, highly scalable, with near real-time concurrent reporting and analysis of multiple physiological parameters, and making this information real-time accessible to healthcare practitioners.

2. Description of the Related Art

Patient monitors and medical monitoring systems include monitors designed for in-patient monitoring and telemetry systems. The telemetry group of systems is aimed at short-haul and local area monitoring. The technology typically targets monitoring ambulatory patients within a hospital campus. The telemetry group feature set provides real-time data feeds, real-time arrhythmia analysis, and near real-time alarms (less than three seconds). Battery life in the patient-worn devices averages between twenty-four to forty-eight hours (using heavy and expensive nine-volt alkaline cells). The area of coverage is usually restricted to a campus, but is sometimes extended to nearby hospitals and clinics via dedicated T1 and T3 telephone lines, and occasionally line-of-sight wireless bridges.

In-home patient monitoring systems include solutions that can be categorized as home-based telemedicine programs. The telemedicine group of systems is aimed at taking a limited set of vitals and transferring these to a base station to be forwarded via wired telephony to a single repository at a doctor's office or hospital. These systems are costly, bulky, and do not follow the patient everywhere. These systems can not be considered “always on” systems. Typically, the monitored patient goes to the monitoring station once or twice a day and works with the equipment to take a set of vitals that are then transferred back to a database.

Improvements to these systems still require up to three wireless hops to get data back to the central station, and battery life is usually less than twenty-four hours. In addition, the sensor normally needs to be discarded upon battery exhaustion, which creates an ongoing expense over the lifetime of the system. Proposed next generation systems aim to use a single server to host connection to roaming client devices, however, these systems have not appeared on the market.

Some examples of these systems include:

Bornn et al., U.S. Pat. No. 5,564,429 discloses a cardio-respiratory alert system for monitoring in a variety of locations, comprising of a patient unit, a base station and remote unit. The remote unit may include a pager carried by a health care professional. The patient has the option of canceling potential alerts and events prior to their transmission to the remote unit. Patient communication also includes a vocal component via a two-way voice link.

Groff et al., U.S. Pat. No. 6,102,856 discloses a wearable medical monitor that continuously monitors and immediately reports any abnormal conditions. The device includes a voice link to a remote user, as well as a ground positioning satellite locating system should the wearer be unable to communicate.

Krausman et al., U.S. Pat. No. 6,306,088 discloses a medical monitoring and recording system that samples data at prescribed time intervals, and time-stamps and transfers the data to an external computer for processing. The device is capable of monitoring various physiological parameters, including those associated with sleep disorders.

Schulze et al., U.S. Pat. No. 6,443,890 discloses a wireless medical monitoring system that periodically samples the patient's medical condition, compares the data to preset parameters, and sends the information to a remote location over a wireless network. The device can also receive interrogation and instruction from the remote location. The device also allows a user to hit a panic alert to notify a health care professional of an emergency condition.

West et al., U.S. Pat. No. 6,544,173 discloses a wireless medical monitoring system that includes a wireless patient monitor that continuously collects patient data and transmits the data to a central monitoring station. The patient monitor may be operated as a standalone, or as part of a network through the choice of the patient.

Sackner et al., U.S. Pat. No. 6,551,252 discloses a medical monitoring system that includes a garment-style monitor unit and a central repository for receiving, storing and processing the patient data. Also disclosed is the ability of the wearer to input data or instructions to the monitor unit. The unit can also contain parameters allowing it to recognize significant physiological events and alert the wearer. Additionally, the wearer may input information indicating compliance with instructions received from the unit, or current symptoms. The patent also discloses that health data may be transmitted to health care professionals via telephone, email, pager, etc. Further, wireless communications may be used to transmit the data.

Phipps et al., U.S. Pat. No. 6,579,231 discloses a portable medical monitoring unit and system that provides for continuous monitoring and recording, as well as real-time notification of abnormal conditions. The device may also include the capability of alerting health care professionals automatically via a wireless network, and provide them with the wearer's location via a global positioning satellite coordinate stored in the device. The device may also be configured to dispense medication.

Linder et al., U.S. Pat. No. 6,681,003 discloses a medical monitoring system that includes a wearable monitor that may continuously record patient data and transmit that data to a central location. The information may then be viewed by individuals via the internet.

Mault, U.S. Patent App. No. 2001/0044588 discloses a medical monitoring system that allows a health care professional to monitor the data at a remote location. Continuous monitoring by the device allows the health care professional greater access to data over a specified time period.

Schulze et al., U.S. Patent App. No. 2002/0019584 discloses a medical monitoring device and wireless system. The device may continuously monitor the patient's parameters, and the monitor has event monitoring capability as well. Alarms may cause the monitor to automatically contact health care professionals, and the patient may also utilize a panic button to call 911. The monitor may communicate with a remote station via a wireless network and the internet.

Mault, U.S. Patent App. No. 2002/0028995 discloses a pregnancy monitoring device and system. The application discloses that the device communicates through a PDA to a computer system, and then to a health care professional via the internet. The application also discloses that the entire system may communicate via a wireless network.

Lang, U.S. Patent App. No. 2002/0118112 discloses a medical monitoring device and system that continuously or batch uploads medical parameters to a central computer by means of a wireless communication device. The information is stored on the central computer in order to provide patient history and data trends, and may be uploaded at designated time intervals or on demand.

Linder et al., U.S. Patent App. No. 2002/0181680 is merely the published application that has matured into U.S. Pat. No. 6,681,003 noted above.

Kumar et al., U.S. Patent App. No. 2002/01198473 discloses a network-based system for remote medical monitoring utilizing the internet that allows for real-time monitoring of a patient. The application discloses that alerts based on the data may be sent to a health care professional, and that the data may be saved in a storage device.

Korth et al., U.S. Patent App. No. 2003/0009088 discloses a patient monitoring system that may utilize a mobile phone to transmit health data over a wireless network. In addition to real-time monitoring, recording of data may occur at specified time intervals or upon an event.

Goldberg, U.S. Patent App. No. 2003/0073884 discloses a portable medical monitoring device and system that receives data via sensors and then displays statistical summaries of the data received. A remote computer may be used in the storage and processing of the data. The remote computer may receive the data via a wireless network and may also be connected to the internet or other network, or comprise a web server.

Quy, U.S. Patent Application No. 2004/0030226 discloses a device and system for wireless medical monitoring that allows wireless access to a patient's health monitoring device by a health care professional. The system may be interactive in that it may include stored parameters, query the patient, and accept input via keypad, audio or visual means. The health monitoring device may be used in the context of a health problem, or an exercise program.

### Short-Comings and Issues in the Prior Art

Major short-comings of the existing solutions include systems that are not highly scalable, data integrity that is not assured, multiple wireless hops and use of intermediate base-stations, and users not always in contact with the monitoring station.

What is meant by failing to be highly scalable is that sensors and patient-worn devices collect data at high rates and produce tens of megabytes of data per day per device (a 3-channel ECG collecting 256 SPS produces ˜67 Mbytes/day). To monitor tens of thousands of patients or more would require communications bandwidth and storage capacity in excess of what is commonly available or affordable today. As for data integrity, data is viewed as a continuous stream of data and no concept of “logical records” is apparent in the current designs. This means that partial loss of or failure of the system causes data loss and a potentially unrecoverable situation for the application software. The use of intermediate base stations induces restrictions on the distance a patient can wander from the unit without severing the connection. Further, the patient-worn devices tend to have limited storage and are not capable of storing extended periods of data when out of reach with the sending unit or base station.

Other short-comings of existing solutions include short battery life, limited on-device storage, high cost of acquisition and operation, and complexity of use.

Some systems have tried to address the hookup portion of complexity by producing a unified pre-molded sensor harness to simplify patient hook-up. The failure of this design is that the design requires a medic to stock several sizes of the harness to accommodate male versus female torsos and children versus adolescents versus adults.

Present solutions do not address today's privacy and security concerns. Existing solutions do not audit, track access to the data, nor ensure the integrity of the data through its entire lifetime.

The existing solutions do not provide an always-on (24×7) accessible solution that is globally accessible. Existing systems record a limited amount of information locally in the patient-worn device then require the patient to find a location where the data can be communicated back to the server. Many of the solutions provide a base station as this relay point. This deployment model is also problematic in that many can not deal with service outages and have issues going back in time to retrieve data that may have been recorded hours ago (especially in the case where a more urgent alarm condition mandates transmitting current data ahead of the earlier recorded data.)

The existing systems do not act as a single Federated Service. That is, the systems do not perform mutual co-operative processing and share patients and sessions between them. This means that a patient hooked up in Maine is likely to have a difficult time being monitored and contacted while traveling to Florida or worse yet, over-seas. Another aspect to the drawback of this model is that the model is not fault tolerant. A major outage in the single server location can take down the entire monitoring infrastructure and render patient monitoring unusable.

The present systems do not allow full-disclosure recordings to be made at the patient-worn device that can be online queried or submitted for post analysis. Although the Holter devices provide for full-disclosure recording, they are not in constant communication with a centralized server and patient monitoring service.

### BRIEF SUMMARY OF THE INVENTION

### Overview

The present invention comprises a medical monitoring system that brings,the hospital-campus telemetry experience to the patient home. This system is designed to enable effective step-down patient care in the home setting while providing the patient with the freedom to go anywhere and remain “logically” tethered to the system. The system achieves this by being distributed in nature, globally accessible, highly scalable, with near real-time concurrent reporting and analysis of multiple physiological parameters, and making this information real-time accessible to healthcare practitioners.

To meet the goal of monitoring anyone, anywhere, 24×7, the invention hereafter termed the “solution”, is implemented as a system consisting of three principle elements:
1. the service delivery model element (Model),
2. the monitoring server and application software element (System),
3. and the patient-worn device element (PWD).

To truly provide a 24×7 capability and make the solution highly scalable and to shield against catastrophic failure, the server application is designed to be deployed in a distributed manner using a Federated Service approach.

### Features Implemented to Overcome the Short-Comings of Prior Art

The server application is implemented as a web-based Federated Service. This means that a group of deployed systems acts as one cohesive system providing load balancing, patient sharing, fault tolerance, and high-availability regardless of geography.

The server application is implemented in such a way as to accommodate flexible business models. This design uses the notion of a care group to capture the behaviors germane to a group of patients and care providers and allows the care providers to build custom rule-sets and escalation procedures for a group reducing the effort required to monitor related groups of patients. The system architecture is highly flexible enabling a healthcare professional to customize the system to meet individual needs in addition to specifying supplemental or over-riding rules to the care group rule set on a per patient basis. The server and services use secure communications channels, methods, and certificates to provide private and reliable communications with the patient-worn device. The server audits all activity from cradle to grave. The audit includes who accessed what data and when it was accessed along with the credentials that were used to access the data. This built-in auditing behavior can not be turned off and ensures data integrity as well as providing complete audit ability for compliance with US Health Insurance Portability and Accountability Act (HIPAA) legislation.

The concept of the patient session file is introduced. The session file is made up of smaller self-contained sensor strip records and contains a signature block with patient correlating marks, CRC, date and timestamp, and position within the overall recording session (see FIG. 7 for details). In order to reduce the volume of traffic being sent over the network and to reduce the storage needs, the system introduces the concept of sending self-contained sensor strip records when:
1. certain specified conditions are met,
2. the event button is pushed,
3. as requested by the Rules Engine.

The stream of periodic sensor strip records enables the application of statistical methods and trend analysis against the collected patient data. The resulting trend analysis and probability information can then be used to predict pending medical events and trigger pro-active care avoiding pending patient trauma and emergency room visits. The flexible data form provided by sensor strip records allows for prioritization of transfers. For example: In a given situation, data is being transferred from the PWD to the system and suddenly the data becomes undeliverable and begins to be queued within the PWD. Meanwhile, a while later, an urgent event occurs requiring data to be sent to the host immediately. As the user returns to the wireless service area, data transmission to the system resumes. The PWD assigns the current event data a higher priority over the queued data and the sensor strip record containing the event is sent to the system first (ahead of the earlier time-stamped data). This higher priority data, as a self-contained sensor strip record, is then processed immediately even though this data is out of time sequence and has a gap between itself and the predecessor strips.

The patient-worn device (PWD) has adaptive behaviors and operates in a pre-determined manner based upon the sensor set plugged into it. Further, the PWD is capable of concurrent voice and data channels allowing a healthcare practitioner to communicate with the patient while the server retrieves sensor strip records from the recorder portion of the device. The PWD also includes an extra sensor channel with an omni-directional microphone enabling the recording of the patient voice during events in order to provide a time-synchronized diary with the physiological measurements.

The server uses client-adaptive and media-adaptive output techniques to automatically tailor out-going traffic to meet an end-users preferences and device capabilities. That is, screen I/O is appropriately tailored for a Cell Phone, PDA, laptop, notebook, or workstation display as appropriate.

### BRIEF DESCRIPTION OF THE DRAWINGS

These and other features, aspects and advantages of the present invention will become better understood with reference to the following description, appended claims and accompanying drawings where:

FIG. 1—Service Implementation & Service Model Overview illustrates the modular and distributed nature of deploying the patient management service. The diagram shows the major players and their relative positions within the solution.

FIG. 2—Federated Service Deployment Detail illustrates the intended implementation detail of a distributed and redundant monitoring service.

FIG. 3—Relationship of Key Roles and Key Components presents an enumeration of the major entities and their relative positions within this solution.

FIG. 4—Server Architecture illustrates the overall architecture and implementation detail of the distributed server software.

FIG. 5—Care Group illustrates the Care Group concept with group member and escalation rules relationships.

FIG. 6—Patient-Worn Device Architecture illustrates the overall architecture and implementation detail within the patient-worn device.

FIG. 7—Modular Data Record Details illustrates the implementation details of the modular and flexible sensor strip record scheme.

Table 1 —Definition of Terms.

### DETAILED DESCRIPTION OF THE INVENTION

### Service Delivery Model Element

The implementation of this solution begins with the service delivery model element. The model element has two key aspects, the human aspect and the machine aspect. The human aspect is multi-faceted whereas the machine aspect is a single facet formed by the intersection of interaction and equipment. A successful service delivery model requires both the human aspect and the machine aspect of the model.

In the many facets of the human aspect, two of them seem more important. These facets are the medical care team that provides the initial patient contact, ongoing patient monitoring, and expert services and the remote care team or visiting nurse facet that provides the personal touch and link between the patient, the equipment, and the medical monitoring team. Successful service delivery depends on a symbiotic relationship between the medical care team, the remote care team, and the patient. Studies have shown that people need human interaction and personal hands-on care in order to increase the odds of successful rehabilitation and post-traumatic care success. This multi-faceted model provides all those components.

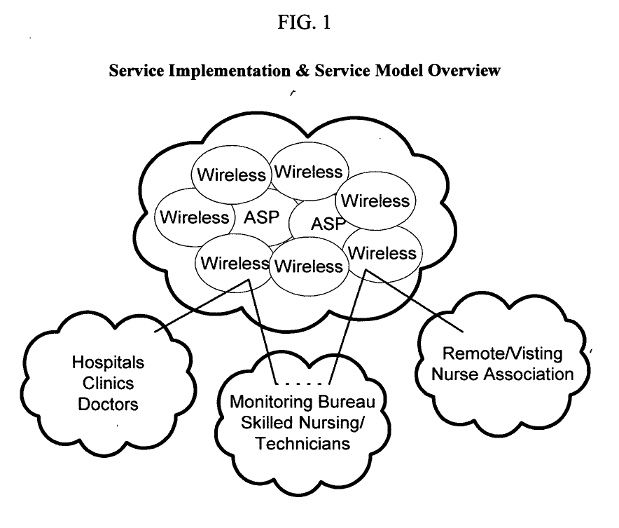

The equipment and services aspect breaks the facets into the access workstations, computer and application hosting providers, and the communications providers. Since a number of insurance companies, hospitals, and businesses all insist on hosting their own servers and another faction of these same entities loathes having anything to do with servers and applications hosting in any way, a model needs to be flexible enough to accommodate all these scenarios concurrently. How is this done? The application is implemented as a mutually co-operative service that can reside in a host, in a server farm, or in a mix of these systems across the Internet. This flexible architecture enables the various entities participating in a patient monitoring program to pick and choose the roles they wish to fulfill. A hospital, for instance, can choose to have its own server farm and host its own copies of the server application while another hospital may deem it too expensive and labor intensive and out-source to an ASP or a partner hospital. As evidenced in FIG. 1, the communications aspect is whatever the market will bear. The solution expects that various telephone companies and wireless carriers exist in the “network mesh” to provide the needed bandwidth and connectivity. By allowing for any mix of players, the solution allows for the creation of a competitive space for the various infrastructure components. This means that no single player can hold the market hostage for a proprietary or closed deployment of a solution. The open architecture also fosters moving the various components to those who are most capable of providing a particular vein of the solution for the most cost-effective price.

The FIG. 1 diagram exhibits the flexibility of the system. In turn, this flexibility drives the need for a workable model to be predefined and built into the server application so that the software is usable out-of-the-box. The solution delivers a predefined implementation instance consisting of predefined roles, rules, and user templates. Further, the server application contains a rules engine that is configurable in terms of patients, patient data, healthcare practitioners, healthcare facilities, monitoring criteria, escalation policies, etc. A typical service delivery model with a corresponding software configuration is pre-constructed so that a successful service can be brought up with minimal effort in a reasonable amount of time by an unseasoned team.

### System Element

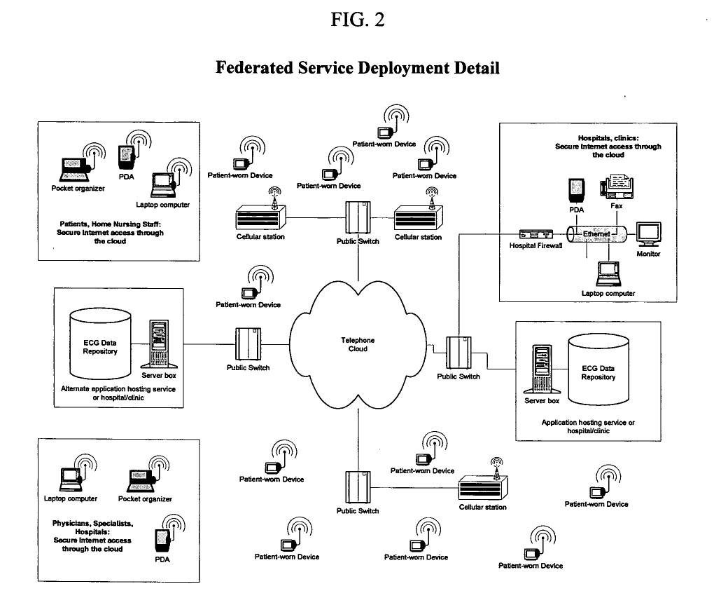

The Server Application element of the solution is implemented as a highly-scalable, highly-available Federated Web Service. What this means is that the Server is designed to be deployed as a distributed resource (in FIG. 2 two instances of the server are shown). The Server accomplishes being a distributed resource by communicating with its peers throughout the Internet as the peers are discovered. Once known to each other, the peer servers propagate common information to each other using a well-known set of services and interfaces. In order to facilitate deployment of other servers and ancillary services, a Server makes its interfaces and service information available to its peers via a world-web-wide accessible directory service such as that provided by UDDI. This open directory mechanism eliminates the need for prior knowledge of IP addresses, URLs, and interfaces. This scheme means that deploying the Nth server is no more complex than deploying the first server.

By having the Servers communicate with each other and act as a singular entity, the Server co-operatively processes and handles patients and monitoring sessions. The redundant roles in this service model decrease the chances of a catastrophic failure taking down the entire monitoring environment. The model allows for a world-wide group of server farms to exist as a single entity if so desired but does not force a Server to be part of the cluster. Since roles and cluster members are elective, an organization could choose to deploy its own Server as a stand-alone entity and have no interaction with the other Peer Servers available on the web.

Refer to FIG. 2 again and note that the public telephony and Internet cloud are at the center of the model. The high availability phone service and Internet resource are central to the model. Moving out a layer in the diagram, note that the next tier of service providers are wireless carriers and application service providers. These two entities are instrumental in providing cost-effective reliable infrastructure for small, medium, and large business enterprises. This solution's model plays off of these key strengths and uses the extensive background and experience of these providers to fulfill the next part of the mission critical role.

With ASPs, wireless carriers, and telecos, providing a solid and reliable infrastructure, we move to the next group of specialists within the model-the medical care providers and their infrastructure. Combining the reliable core infrastructure with the industry specialists allows the hospitals, clinics, doctors, nurses, and other practitioners to focus on the work of caring for the people rather than equipment and software. Lastly, medical facilities and healthcare practitioners can use any ordinary desktop, laptop, PDA, cell phone, or other electronic device to access data and system services allowing these users to use their own familiar equipment and focus their attention on delivering medical care.

### Patient-Worn Device Element

The patient-worn device or PWD is the final integral part of the solution. The system relies on the PWD to maintain data integrity and a permanent record of all bio-sensor data that is acquired via its various attached sensors. The model element maintains the PWD as the focal point of the solution since it is the direct link to the patient.

### Service Delivery Model Details

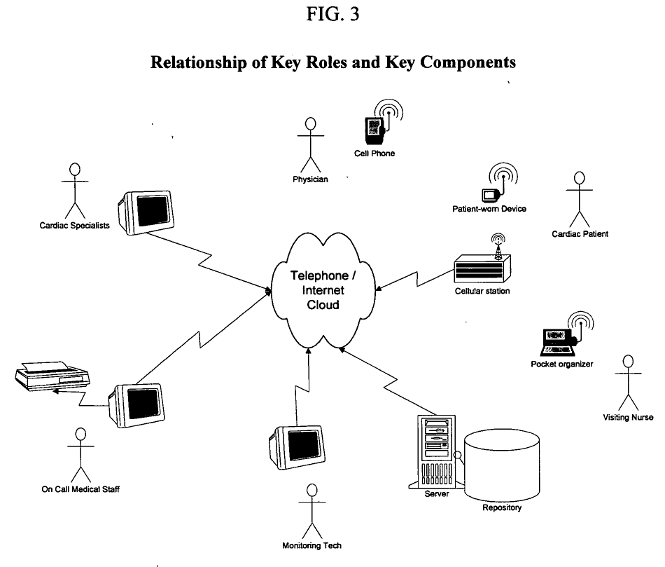

There are several major roles in this solution as well as key components. In the next section, we answer the questions: Who are the players and what are their roles? What are the top-level components and where do they fit in the picture? Refer to FIG. 3 as we answer these questions and assume that we are deploying the more robust deployment model with multiple providers and multiple resources in play.

### Key Players and their Roles

One of the first key roles is the application service provider or ASP. The ASP is tasked with providing a UL-approved highly-available equipment space with reliable servers. The ASP takes into consideration the physical plant and infrastructure security issues including data backup, fail-over plans, backup power, backup telephone/Internet service, 24×7 staffing, etc. The ASPs role is to ensure that the clients can just assume that when they want to access the servers that the servers are always available.

The next set of major players and roles are often taken for granted. The roles are those of the telephone carrier, wireless carrier, and Internet Service Provider. This solution assumes that there are service contracts with at least one major entity in each of the mentioned roles. This enables responsibility and accountability to be assigned to a specific entity.

With a sound and highly available infrastructure in place, the next roles are those of the medical providers. The primary entity in a monitoring situation is the cockpit or monitoring group. It is this group that performs the day-to-day operations on managing the operation of the software and watching the patients and handling the alarms and escalations. Another key role in the model is the visiting nurse or remote medical professional. The visiting nurse role is the one that facilitates success in monitoring. Inevitably, there are equipment issues and patient issues. It is the visiting nurse that checks up on the patient in person to ensure that all is well. The nurse can assist with battery replacement, sensor lead issues, operational issues, or just plain patient concern. With a good deal of technology in the loop, it is important to keep a person in the loop, but at the same time minimize the activity required of the visiting nurse. Other roles include the hospital, clinic, doctor, specialist, and on-call staff. In the event that escalation rules fire, there needs to be a group of available resources to handle the patient escalation.

The final role is that of the patient. The patient is diagnosed with a condition and released from the clinic or hospital and requires further observation. The doctor prescribes an order for hookup and evaluation and so the patient becomes connected to the system via the patient-worn device. The patient is coached by technicians on the availability of the two-way communications on the PWD and is also told about the emergency transmission or EVENT button.

### Key Components and their Position in the Solution

The Server is the main ingredient of the automation solution. The combination of the hardware and software form the server entity.

The telephone switch network, wireless switch network, and Internet backbone network are the major elements, but they are rather difficult to pin down. It is sufficient for this discussion to state that the entities exist, they have roles, and they are needed to complete the solution.

The patient-worn device is the next significant element or piece of equipment in the picture. It is the final element that we have direct control over. One such device is required for each monitored patient along with a sensor set to obtain the requested physiological data.

Other elements that are in play include computers, telephones, PDAs, cell phones, fax machines, and other technological pieces of equipment. As these elements belong to the various members that participate in the solution, the exact nature and mix of the elements can not be specified. As a result, the solution must accommodate this diverse set of needs and requirements.

### System Element Details

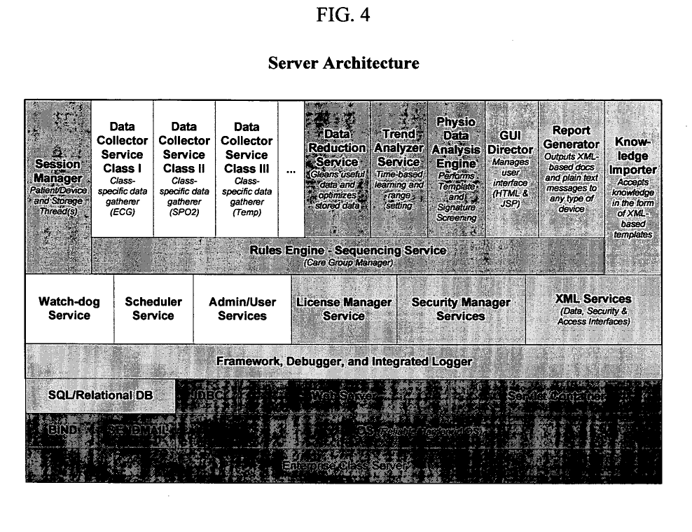

For this discussion, we evaluate the server architecture from the bottom-up. The implementation of this solution is executed in Java, but deployment in other languages is possible. Refer to FIG. 4 for the remainder of this discussion.

### Server Architecture and Implementation Detail

The server infrastructure begins with a commercial enterprise-class server (such as a Sun E-Series Server) with a reliable and security hardened operating system. Additional services including an email server and Internet Domain Name Service are required to provide message deliver, message reception, and other network-centric services.

Next up are the Apache Web Server (or equivalent) with the Apache Axis Web Services plug-in, the Tomcat Servlet Container (or equivalent), and a SQL/Relational Database engine with a JDBC connector. This layer provides the Internet Application layer along with the services needed to create persistent data in a database and serve and store data from the Internet users and patients being monitored.

Moving up into the application space introduces the framework, debugger, and integrated logger. The framework provides the base classes, scheduling, synchronization, auditing, and other common components needed to provide a robust application. The debugger and logger are integrated in this layer to provide fundamental troubleshooting and auditing from the outer-most functions to the inner-most core workings of the solution.

The next layer introduces functionality that lives close to the core but differs in that this layer contains components that are slightly exposed to the user. Components found in this layer include the watchdog service that continuously monitors the health of the application, the network connections, and the server itself. The next components include the scheduler, and user and admin services. These components control the availability and scheduling of the solutions resources. Next up are the license manager ensuring that a specific instance of a server provides only the services commissioned and authenticated for that server. The security manager exists to ensure roles, privileges, behaviors, and user actions are consistent with the defined system policies. Choosing the Federated Service as a fundamental part of the solution imposed various protocols and transports including HTTP and SOAP. The Federated Service selection further drove the; need for XML-based services to stay consistent with the remainder of the solution model. The XML layer adds important capabilities to the solution. The XML layers allows the solution to readily import, export, and exchange users, rules, knowledge, and data with other disparate systems including those that support the electronic medical record.

Next, we move up another layer and encounter the central nervous system of the solution. The abstraction at this layer is termed the care group. The care group is a related group of services, interfaces, and entities that provide the central make-up of the rules processing engine. When rules processing is intersected with the various components and modules that live above it, a robust system evolves that yields a good deal of capability. More about the care group and rules processing is covered in the next section.

The layered model description is completed by enumerating the high-level services that ride on top of the previously described infrastructure. It is these top-level services that provide the visible interfaces, behaviors, and capabilities of the solution. The components are discussed traversing the diagram from right to left starting with the Knowledge Importer. The Knowledge Importer is the component responsible for bringing new rules, templates, measurements, and other related material into the solution. These knowledge components are used later by the system to construct policies, monitoring criteria, and escalation rules which are in-turn captured under the notion of a care group. Next up are the GUI Director and Report Generator. The Report Generator is responsible for formatting and delivering data from the system to users in terms of screen output, printed reports, and XML-based documents for machine consumption. The Report Generator is closely related to the GUI Director in that the GUI Director is the fundamental component that drives screen-based input and output. The GUI Director provides an abstraction of the user interface layers such that device independent presentations can be formulated. Since screen real estate various by device class, the GUI director can, with a rule-set, reformulate various data so that it can be presented to the user in a usable form without the user having to manipulate their smaller device screens just to look for a simple message or measurement result. The next group of services is a trio of related services. These are the Data Reduction Service, the Trend Analyzer, and the Physio Data Analysis Engine. The Data Reduction Service takes incoming streams of data and attempts to reduce the streams into a series of related streams and provides a statistical basis by sorting and counting various aspects of physiological sensor data. The Trend Analyzer establishes dynamic thresholds, then measures and compares various aspects of the signals to the dynamic threshold and pre-stored trigger points in the care group. The Trend Analyzer trains itself over time and becomes a good predictor of what is expected and unexpected behavior. The ability to dynamically detect deviations in behavior and feed them to the Rules Engine and Analysis Engine are crucial. The Analysis Engine uses a combination of abnormality templates and signal processing to comb through data to determine if detected deviations in measurements is normal or requires further analysis by a human-in-the-loop. Any trigger outputs of this trio cause the Rules Engine to execute rules and perform the escalation actions as defined and stored in the care group. Next in the list are the Data Collection Services. Each Data Collection Service is tuned to a specific physiological sensor. For example, one collection service acquires, normalizes, and processes cardiac data, another collection service handles temperature measurements, and yet another handles blood pressure measurements. During system operation, these data collection service components constantly acquire new data from their respective sensors and then normalize this data so that it can be fed into the statistical and analysis portion of the system with known acquisition rates and known linear measurements in pre-determined units. An example of an adjustment that might be performed by a data collection service is a PWD sensor that acquires cardiac rhythms at 512 samples per second and this data needs to be adjusted or bridged to the sampling rate of 128 samples per second in order to fir the scheme and templates supported by the analysis engine. These collection modules are also responsible for ensuring data integrity and packet re-assembly. The various sensor-specific modules ensure that the data arriving is complete and valid. In addition, the sensor-specific modules generate requests to various sensors to re-transmit corrupt or missing data needed for an evaluation cycle. This relieves the other layers of the burden of dealing with un-normalized, incomplete, or damaged data. The last service in this tier is the session manager. The session manager operates in conjunction with the various sensor-specific modules and is responsible for managing all of the monitored patient streams and sorting/validating the various data blocks and records. These modules are responsible for data integrity and for requesting the re-transmission of missing/damaged blocks necessary to re-construct a sensor strip record.

### Care Group Detail

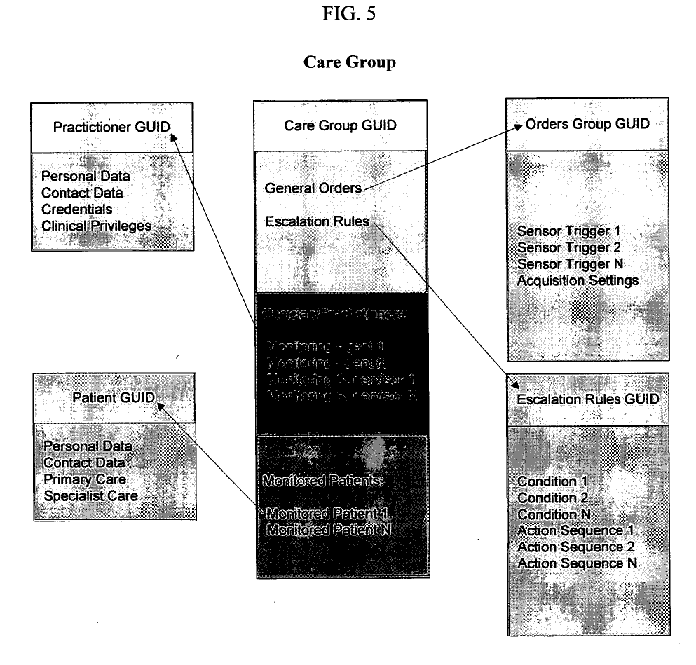

As illustrated by FIG. 5, the Care Group is the fundamental entity used in this solution. This entity captures the knowledge of who is monitoring whom for what conditions and when. It also captures the knowledge about who notifies whom and under what conditions the notifications occur. The escalation policies, the form of notifications provided, and all of the inter-action of the players is orchestrated by the Care Group.

The central entity Care Group contains the unique identity of an instance of the group and the general monitoring constraints that are assigned to a patient by default. During a patient hook-up sequence, a technician adds a patient to a Care Group. As a result of being admitted to a Care Group, the patient is provided with a default monitoring group, a default set of monitoring criteria, and a default escalation and notification policy. The technician can then make custom modifications to the monitoring orders as directed by the patient condition and/or physician order. These per-patient customizations are captured in a separate location so that the general group rule set and conditions are not altered. Only the rules that apply to the entire Care Group are contained directly by the Care Group entity. The next facet of the Care Group entity is the list or roles and people assigned to fulfill those roles. The roles belong to the Care Group entity, but the actual information about the individuals fulfilling the role is contained in the health care providers entity. The next section of the Care Group entity relates to the patients being monitored. The patient section contains references to the patient entities that are members of the Care Group just as the healthcare providers section does. The actual details describing the patient and other pertinent patient data are captured in the patient entity itself. The next section of the Care Group entity contains references to all the various rules and procedures that apply to the Care Group. The various rules and thresholds are contained by the rules entity. The knowledge surrounding a Care Group is captured by an instance of the central entity but the relationships and related entities carry the details of these relationships.

The system can also be described as a collection of free-running event-driven classes that respond to data reception and user requests causing the processing of information to occur. The processing consists of actions such as data collection and normalization, data scanning, rules processing, trigger generation, condition reporting, email notifications, and placing of phone calls. The eventual result is the visiting of patients by practitioners and the recall of patients to appropriate care facilities as needed. It is the robust and programmable interaction of the classes that makes the solution what it is-a truly dynamic and adaptable system.

### Patient-Worn Device Detail

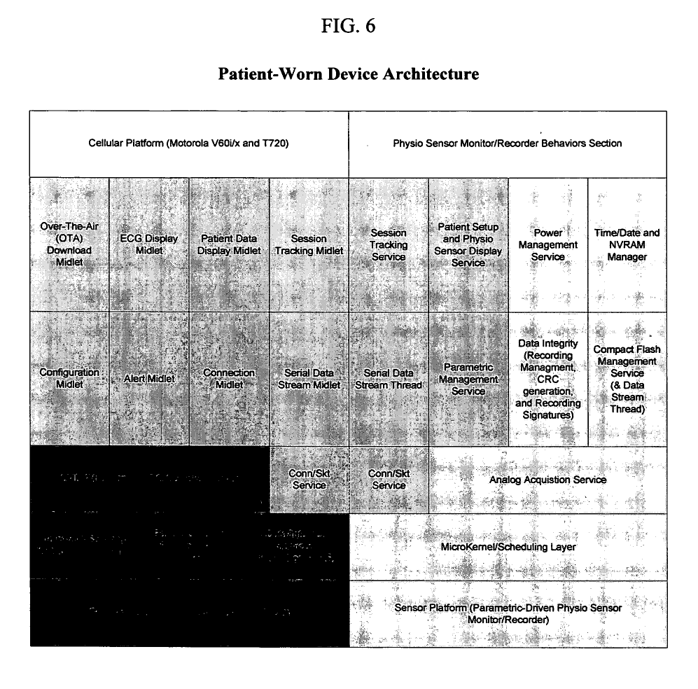

The patient-worn device or PWD is an integral part of the solution. The system relies on the PWD to maintain data integrity and a permanent record of all bio-sensor data that is acquired via its various attached sensors. Refer to FIG. 6 for a detailed block diagram of the PWD architecture.

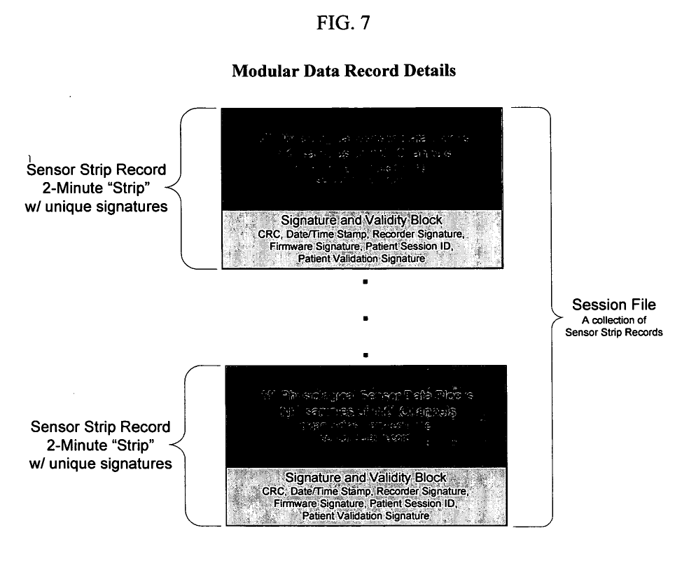

The PWD can be programmed to send periodic data records to the server for abnormality analysis and trend analysis. The PWD can also be programmed so that various simple trigger conditions deliver a collected data record (see FIG. 7 for the structure of the record) to the system for storage and analysis.

Conceptually, the PWD consists of two distinct logical parts termed modules. The first module compromises the biomedical module and is tasked with data acquisition and the handling of the physiological sensors. The second module is the communications module and is tasked with acting as a proxy and interfacing and handling the communications between the biomedical module and the Server. While the present implementation provides the two modules as separate entities connected via a communications interface, the design is not constrained to this implementation model. These two groups of functions can be combined into one device or they can be split into more entities. For example: The fundamental device can be offered as a single unit but various sensors can be deployed via wireless means over different regions of the body. A device solution delivered in this way could easily consist of several small bio-sensor elements and the transceiver/collector unit. While the multi-sensor deployment model positions sensors in the most useful spots and eliminate cabling, it does so at the cost of increasing patient hookup time and hookup complexity.

Some of the key elements in the biomedical module are the power management module and the real-time clock module. The battery-operated unit has its own power source and ability to keep accurate time. As the unit derives its own time, and the data is collected based upon intervals of that time reference, there is no notable error in the collection and time-stamping of the collected data.

Other key elements in the biomedical module include the data integrity manager and the on-chip storage state machine. These two elements ensure that data is organized and stored in the proper strip record format and that the integrity and quality of that data can be guaranteed.

The parametric monitoring and measurements module exists on the biomedical module as simpler measurements can be made more frequently in real-time to help determine if and when strips meet various trigger conditions. Upon detection of the various triggers, the parametric module can schedule a strip record for delivery to the Server for further analysis and trend analysis.

The communications module does not have to be integrated into one physical package. The communications module can take the form of a cell phone and can be paired with the measurements module via a communications port and the communications firmware.

Session control, data transfer, alerts, configuration, and other basic software modules exist on the communications module. A few of the modules can exist on the PWD however they are organized as part of a suite that can also be downloaded to a doctors PDA or cell phone using the over-the-air download functionality. This decision and practice allows the physician reviewing data from a PWD to receive an identical copy of the display drivers as well as the data from the PWD. What this means is that what you see locally at the PWD the doctor also sees remotely on his personal computing device. This practice reduces, if not eliminates, the issue that arise of taking data meant for display at one scale and trying to display it at another with unknown properties. By taking the display issues out of the equation, we improve data delivery, analysis, and hopefully improve the outcomes.

### Modular Data System Details

The most obvious short-comings of past systems are the need to have consecutive data with no missing segments and a continuous feed of that data to the server. This creates a large amount of data requiring a great deal of bandwidth and introduces the issues of where to look in the data and when to look at the data. This situation also leaves an unresolved issue of how to detect and deal with missing segments of data.

To begin addressing these short-comings, this solution introduces the concept of “strip” segments. The system is modeled after the paradigm that physicians and cardiologists have relied upon for decades, a collection of short strips taken at regular intervals and analyzed to produce a trend that the physician can compare against an accepted standard. Building upon the strip notion, we add the signal processing capability, the mathematical assessment capability, and the ability of the patient to send in impromptu data sets. Now we have a very robust data collection scheme that has excellent predictability and statistical classification abilities.

Now that we've created a fundamental “strip” record, the length of this record is “tuned” to match the current medical and cardiologists' model. Although any length will suffice, we select two minutes as the desired strip length. At this time, two minutes is critical as a choice because the currently available data models and statistics are based on analysis of decades of two-minute strip samples. For the time being we will use this segment length, however, the length can be dynamically adjusted once more data from new models becomes available.

The next step in the process is to encapsulate and validate each strip as a stand-alone entity. As data is processed in a single rule cycle, a stand-alone strip is evaluated without regard to any other segment. The strip segment is validated with a date and time stamp and a CRC record to ensure data integrity. Further, the strip record contains the PWD serial number, firmware version number, assigned patient alias ID, patient uniqueness identifier (currently the date-of-birth) along with other session pertinent data. What this collection of data provides is a complete data sample that can be analyzed on its own merit. The sensor strip record also provides a set of identifiers guaranteed to differentiate a strip should part of the header become corrupt for any reason.

When a consecutive series of these records is taken into consideration, an unbroken or continuous data file can be constructed for evaluation. Given that the data is periodic in nature and stored in consecutive periodic pieces within the PWD, the analysis system is free to take samples received on a periodic basis and make some calculated assessments of the patient condition based on statistical models. Sine the device contains a full-disclosure recording, the system is also free to request any number of segments with any desired interval spacing for further analysis. Lastly, escalating a patient condition to a physician or specialist for evaluation could trigger a more thorough review of the patient data which can then be retrieved from either the local database or from the PWD on-board storage module. It is important to be able to retrieve any segment from the full-disclosure recording because a doctor might, for instance see abnormal physiological measurements or a heart arrhythmia and decide he wants to review the patient condition for 10 minutes leading up to the event. Even if the system were programmed to report only on 30 or 60 minute intervals, the physician could request segments of a recording that were not present in the database and the request would be automatically deferred to the patient-worn device. This would then provide the detailed data on demand and avoid congesting the network with continuous feeds.

Although the present invention has been described with reference to particular embodiments, it will be apparent to those skilled in the art that variations and modifications can be substituted therefore without departing from the principles and spirit of the invention.

TABLE 1
Definition of Terms

| | |
|---|---|
| Care Group | The collection of software-derived entities used to |
| | define the healthcare practitioners, monitored |
| | patients, rules, and parameters that comprise a |
| | monitored group of medically-related patients. |
| Event Button | A button that is in communication with the patient- |
| | worn device that a patient can activate to ensure a |
| | sensor strip record is sent immediately to the help |
| | desk when they don't feel well. The event button |
| | also causes the data in the sensor strip record to |
| | be super-imposed with an event marker indicating the |
| | time and physiological conditions present when the |
| | patient didn't feel well. |
| Event Marker | A unique modulation scheme used to modulate sensor |
| | strip record data to high-light an abnormal situation |
| | in the patients condition. The marker is usually |
| | inserted by the patient depressing the event button |
| | on the patient-worn device. |
| Federated | A group of two or more servers or server farms acting |
| Service | as a single entity providing a distributed application |
| | with high availability and failover capabilities. |
| Patient-Worn | The device worn by the patient that comprises the |
| Device | sensors, sensor data collectors, sensor data |
| | recorders, and transceiver. |
| Rules Engine | The logic processing entity at the heart of the data |
| | processing system. The rules engine allows |
| | measurements to be made and handled according to |
| | custom business rules as they apply to each set of |
| | conditions, users, patients, and circumstances. |
| Sensor Strip | A stand-alone file fragment usually two minutes in |
| Record | length that contains a collection of sensor data |
| | sampled at some regular interval. The strip is |
| | stored, validated, and correlated to a specific |
| | patient and can be combined with other fragments to |
| | make up samples that cover greater time periods. |
| Server | The term Server refers to a logical instance of the |
| | application server platform. This can refer to either |
| | an individual server or a server farm. |
| Server | The Server Application refers to the software that |
| Application | makes up the federated service and runs on the Server |
| | platform. |
| Session File | A session file is a collection of related Sensor |
| | Strip Records. |
| Solution | The term solution in this document refers to the |
| | combination of the device, hardware, software, |
| | and business model that make up the patent filing. |
| System | The combination of a Server and Server Application |
| | form to make an instance of a complete System. |

What is claimed is:

1. A method for remotely monitoring a patient, comprising:
Accessing an application service provision system;
Defining a care group within said application service provision system, wherein said care group comprises at least one health care practitioner;
Creating a patient profile for said patient within said application service provision system;
Assigning said patient to said care group;
Evaluating said patient at a healthcare facility;
Specifying orders to a patient-worn device, wherein said orders are at least partly based on the patient evaluation;
Communicating said orders to said patient-worn device;
Hooking up said patient to said patient-worn device;
Monitoring said patient in accordance with said orders, wherein patient data is generated and stored locally;
Transmitting patient data to said application service provision system;
Validating said patient data;
Accessing said patient data;
Analyzing said patient data;
Performing health care actions, wherein said health care actions are based at least in part on the analysis of said patient data.

2. The method of claim 1, wherein said application service provision system is a federated service.

3. The method of claim 2, wherein said health care practitioner comprises at least one doctor.

4. The method of claim 3, wherein said health care practitioner comprises at least one nurse.

5. The method of claim 4, wherein said health care practitioner comprises at least one health care technician.

6. The method of claim 2, wherein said step of defining a care group further comprises assigning a group rule set, assigning at least one patient, and assigning at least one health care practitioner to said care group.

7. The method of claim 2, further comprising the step of communicating with said patient by said health care practitioner.

8. The method of claim 7, wherein said step of communicating with said patient includes voice communication.

9. The method of claim 8, wherein said step of communicating with said patient includes data communication.

10. The method of claim 9, wherein said voice communication and said data communication occurs concurrently.

11. The method of claim 2, wherein said step of accessing said patient data may be performed concurrently by a plurality of health care practitioners in real time.

12. The method of claim 2, wherein said step of accessing said patient data includes communicating by means of an electronic device.

13. The method of claim 12, further comprising the step of auditing said step of accessing said patient data.

14. The method of claim 13, further comprising the step of reducing data.

15. The method of claim 14, further comprising the step of analyzing trends.

16. The method of claim 15, further comprising the step of analyzing physiological data.

17. A remote patient monitoring system comprising:
an application service provision system;
at least one care group defined within said application service provision system;
orders for said care group;
at least one patient assigned to said care group;
at least one health care practitioner assigned to said care group;
a patient-worn device, wherein said patient-worn device is capable of receiving patient data, and wherein said patient-worn device is capable of a remote communication with said application service provision system;
a patient evaluation based on said patient data;
health care actions based at least in part upon said patient evaluation.

18. The remote patient monitoring system of claim 17, wherein said application service provision system comprises a federated service.

19. The remote patient monitoring system of claim 18, further comprising automatic end-point adaptation.

20. The remote monitoring system of claim 19, wherein said patient-worn device further comprises a full-disclosure storage.

21. The remote monitoring system of claim 20, further comprising an auditing means.

22. The remote patient monitoring system of claim 20, further comprising a sensor strip record, wherein said sensor strip record comprises a representation of said patient data.

23. The remote patient monitoring system of claim 22, wherein said sensor strip record further includes a validation block.

24. The remote patient monitoring system of claim 23, wherein said sensor strip record includes a record period.

25. The remote patient monitoring system of claim 24, wherein said record period is substantially two minutes in duration.

26. The remote patient monitoring system of claim 25, wherein said sensor strip record includes an event marker.

27. The remote patient monitoring system of claim 26, wherein said event marker comprises a substantially transparent overlay, wherein said substantially transparent overlay is located on said sensor strip record.

28. The remote patient monitoring system of claim 27, wherein said event marker is created after an event condition delay.

29. The remote patient monitoring system of claim 28, wherein said event condition delay is approximately ten seconds in duration.

30. The remote patient monitoring system of claim 17, wherein said health care practitioner comprises at least one doctor.

31. The remote patient monitoring system of claim 30, wherein said health care practitioner further comprises at least one nurse.

32. The remote patient monitoring system of claim 31, wherein said health care practitioner further comprises at least one remote health professional.

33. The remote patient monitoring system of claim 32, wherein said orders are specified by said health care practitioner.

34. The remote patient monitoring system of claim 33, wherein said patient evaluation is conducted by said health care practitioner.

35. The remote patient monitoring system of claim 34 wherein said health care actions are performed based at least in part on said orders.

36. The remote patient monitoring system of claim 17, wherein said patient-worn device further comprising a plurality of voice data channels.

37. The remote patient monitoring system of claim 36, wherein said plurality of voice data channels further comprise a secure communication means.

38. The remote patient monitoring system of claim 36, wherein said plurality of voice data channels includes a first voice data channel, and wherein said plurality of voice data channels includes a second voice data channel.

39. The remote patient monitoring system of claim 38, wherein said first voice data channel is capable of allowing a first communication, wherein said first communication further comprises an oral communication between said patient and said health care practitioner.

40. The remote patient monitoring system of claim 39, wherein said first communication and said remote communication may occur concurrently.

41. The remote patient monitoring system of claim 40, wherein said second voice data channel comprises a patient voice recorder.
